(19) United States
(12) Patent Application Publication
(10) Pub. No.: US 2024/0358709 A1
(43) Pub. Date: Oct. 31, 2024

(54) METHODS AND COMPOSITIONS FOR TREATING SLEEP APNEA

(71) Applicant: APNIMED, INC. (DELAWARE), Cambridge, MA (US)

(72) Inventors: David P. WHITE, Denver, CO (US); Luigi TARANTO-MONTEMURRO, Brookline, MA (US); Ronald FARKAS, Clarksville, MD (US); Lawrence G. MILLER, Cambridge, MA (US)

(21) Appl. No.: 18/687,660

(22) PCT Filed: Aug. 30, 2022
(86) PCT No.: PCT/US2022/041990

§ 371 Date: Feb. 28, 2024

Related U.S. Application Data
(60) Provisional application No. 63/239,064, filed on Aug. 31, 2021.

(51) Int. Cl. A61K 31/5375; A61F 5/56; A61K 31/138; A61K 31/216; A61K 31/437; A61K 31/496; A61P 11/04

(52) U.S. Cl. CPC A61K 31/5375; A61F 5/566; A61K 31/138; A61K 31/216; A61K 31/437; A61K 31/496; A61P 11/04

(57) ABSTRACT

Methods of treating pharyngeal airway collapse (e.g., sleep apnea) by administering a norepinephrine reuptake inhibitor (NRI) in combination with mandibular advancement device (MAD) therapy are described herein. The treatment may further comprise administration of a muscarinic receptor antagonist (MRA) and/or a hypnotic.

### CROSS-REFERENCE TO RELATED APPLICATION

This application claims priority to and benefit of U.S. provisional application No. 63/239,064, filed Aug. 31, 2021, the entire contents of which are incorporated herein by reference.

### TECHNICAL FIELD

The present invention provides methods of treating pharyngeal airway collapse (e.g., sleep apnea) by administering a norepinephrine reuptake inhibitor (NRI) in combination with mandibular advancement device (MAD) therapy.

### BACKGROUND

Obstructive Sleep Apnea (OSA) is a common disorder caused by collapse of the pharyngeal airway during sleep. OSA can have serious health consequences.

### SUMMARY

One aspect of the present invention provides a method of treating a subject having a condition associated with pharyngeal airway collapse, the method comprising administering to a subject in need thereof an effective amount of a norepinephrine reuptake inhibitor (NRI) in combination with mandibular advancement device (MAD) therapy.

Embodiments of this aspect of the invention may include one or more of the following optional features. In some embodiments, the NRI is a norepinephrine selective reuptake inhibitor (NSRI). In some embodiments, the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof. In some embodiments, the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof. In some embodiments, the NRI is selected from the group consisting of atomoxetine or a pharmaceutically acceptable salt thereof and reboxetine or a pharmaceutically acceptable salt thereof. In some embodiments, the NRI is atomoxetine or a pharmaceutically acceptable salt thereof. In some embodiments, the NRI is reboxetine or a pharmaceutically acceptable salt thereof. In some embodiments, the method further comprises administering a muscarinic receptor antagonist (MRA) to the subject. In some embodiments, the MRA is selected from the group consisting of atropine, propantheline, bethanechol, solifenacin, darifenacin, tolterodine, fesoterodine, trospium, and oxybutynin, or a pharmaceutically acceptable salt thereof. In some embodiments, the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof. In some embodiments, the MRA is oxybutynin or a pharmaceutically acceptable salt thereof. In some embodiments, the MRA is (R)-oxybutynin or a pharmaceutically acceptable salt thereof. In some embodiments, the method further comprises administering a hypnotic to the subject. In some embodiments, the hypnotic is selected from the group consisting of trazodone, zolpidem, eszopiclone, benzodiazepines, gabapentin, tiagabine, and xyrem. In some embodiments, the hypnotic is trazodone. In some embodiments, the hypnotic is zolpidem. In some embodiments, the atomoxetine or pharmaceutically acceptable salt thereof is administered at a dose of from about 20 to about 150 mg. In some embodiments, the atomoxetine or pharmaceutically acceptable salt thereof is administered at a dose of from about 25 to about 100 mg. In some embodiments, the oxybutynin or pharmaceutically acceptable salt thereof is administered at a dose of from about 1 to about 15 mg. In some embodiments, the oxybutynin or pharmaceutically acceptable salt thereof is administered at a dose of from about 2 mg to about 10 mg. In some embodiments, the (R)-oxybutynin or pharmaceutically acceptable salt thereof is administered at a dose of from about 0.5 to about 10 mg. In some embodiments, the (R)-oxybutynin or pharmaceutically acceptable salt thereof is administered at a dose of from about 1 mg to about 5 mg. In some embodiments, the NRI, MRA and/or hypnotic are administered in a single composition. In some embodiments, the single composition is an oral administration form. In some embodiments, the oral administration form is a syrup, pill, tablet, troche, capsule, or patch. In some embodiments, the single composition is in an immediate release formulation. In some embodiments, the single composition is in an immediate release formulation and the NRI is administered at a dose of from about 20 to about 150 mg and the MRA is administered at a dose of from about 1 to about 15 mg. In some embodiments, the single composition is in an immediate release formulation and the NRI is administered at a dose of from about 25 to about 100 mg and the MRA is administered at a dose of from about 2 to about 10 mg. In some embodiments, the single composition is in an immediate release formulation and the NRI is administered at a dose of from about 20 to about 50 mg and the MRA is administered at a dose of from about 2 to about 10 mg. In some embodiments, the single composition is in an immediate release formulation and the NRI is administered at a dose of from about 40 to about 80 mg and the MRA is administered at a dose of from about 2 to about 10 mg. In some embodiments, the single composition is in a controlled release formulation. In some embodiments, the single composition is in a controlled release formulation and the NRI is administered at a dose of from about 20 to about 150 mg and the MRA is administered at a dose of from about 0.5 to about 10 mg. In some embodiments, the single composition is in a controlled release formulation and the NRI is administered at a dose of from about 25 to about 100 mg and the MRA is administered at a dose of from about 2 to about 6 mg. In some embodiments, the single composition is in a controlled release formulation and the NRI is administered at a dose of from about 20 to about 50 mg and the MRA is administered at a dose of from about 2 to about 6 mg. In some embodiments, the single composition is in a controlled release formulation and the NRI is administered at a dose of from about 40 to about 80 mg and the MRA is administered at a dose of from about 2 to about 6 mg. In some embodiments, the single composition further comprises a pharmaceutically acceptable carrier. In some embodiments, the single composition is administered prior to the start of the mandibular advancement device (MAD) therapy. In some embodiments, the single composition is administered concurrently with the mandibular advancement device (MAD) therapy. In some embodiments, the condition associated with pharyngeal airway collapse is sleep apnea. In some embodiments, the condition associated with pharyngeal airway collapse is obstructive sleep apnea (OSA). In some embodiments, the condition associated with pharyngeal airway collapse is snoring. In some embodiments, the condition associated with pharyngeal airway collapse is simple snoring. In some embodiments, the subject is in a non-fully conscious state. In some embodiments, the non-fully conscious state is sleep.

Another aspect of the invention provides a norepinephrine reuptake inhibitor (NRI) and a mandibular advancement device (MAD), for use in treating a subject having a condition associated with pharyngeal airway collapse. Some embodiments further comprise a muscarinic receptor antagonist (MRA) and/or a hypnotic.

Another aspect of the invention provides a therapeutic combination comprising (a) a pharmaceutical composition comprising a norepinephrine reuptake inhibitor (NRI) and a pharmaceutically acceptable carrier, and (b) a mandibular advancement device (MAD) for use in treating a subject having a condition associated with pharyngeal airway collapse. In some embodiments, the pharmaceutical composition further comprises a muscarinic receptor antagonist (MRA) and/or a hypnotic.

Another aspect of the invention provides atomoxetine or a pharmaceutically acceptable salt thereof, oxybutynin (e.g., (R)-oxybutynin) or a pharmaceutically acceptable salt thereof, and a mandibular advancement device (MAD), for use in treating a subject having a condition associated with pharyngeal airway collapse.

Another aspect of the invention provides atomoxetine or a pharmaceutically acceptable salt thereof, a hypnotic (e.g., trazodone or zolpidem or a pharmaceutically acceptable salt thereof), and a mandibular advancement device (MAD), for use in treating a subject having a condition associated with pharyngeal airway collapse.

Another aspect of the invention provides atomoxetine or a pharmaceutically acceptable salt thereof, oxybutynin (e.g., (R)-oxybutynin) or a pharmaceutically acceptable salt thereof, and a mandibular advancement device (MAD), for use in treating sleep apnea.

Another aspect of the invention provides atomoxetine or a pharmaceutically acceptable salt thereof, oxybutynin (e.g., (R)-oxybutynin) or a pharmaceutically acceptable salt thereof, and a mandibular advancement device (MAD), for use in treating snoring.

Another aspect of the invention provides atomoxetine or a pharmaceutically acceptable salt thereof, a hypnotic (e.g., trazodone or zolpidem or a pharmaceutically acceptable salt thereof), and a mandibular advancement device (MAD), for use in treating sleep apnea.

Another aspect of the invention provides atomoxetine or a pharmaceutically acceptable salt thereof, a hypnotic (e.g., trazodone or zolpidem or a pharmaceutically acceptable salt thereof), and a mandibular advancement device (MAD), for use in treating snoring.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

The following figures are provided by way of example and are not intended to limit the scope of the claimed invention.

FIG. 1 is a graphic illustration of an obstructive apnea. The top channel shows the electroencephalogram (EEG) pattern of sleep. The next channel represents airflow. The next three channels show ventilator effort by movements of the rib cage and abdomen and changes in esophageal pressure, all of which reflect a respiratory effort against an occluded upper airway. The last channel indicates oxyhemoglobin saturation.

FIG. 2 is an overview of the MandADO study design described herein.

### DETAILED DESCRIPTION

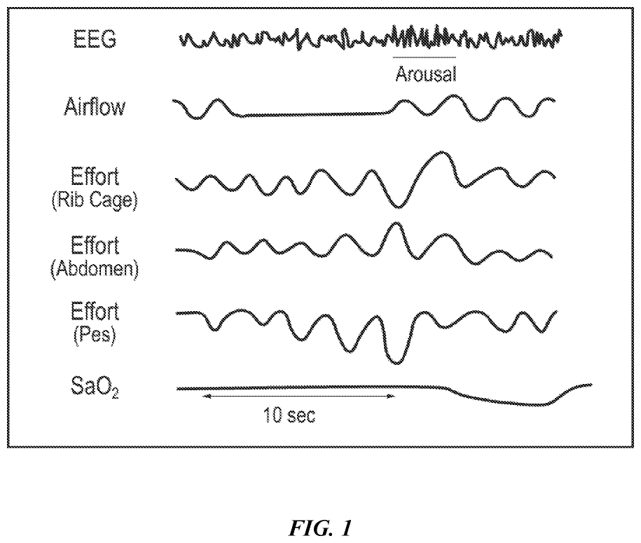

In humans, the pharyngeal airway region has no bone or cartilage support, and it is held open by muscles. When these muscles relax during sleep, the pharynx can collapse resulting in cessation of airflow. As shown in FIG. 1, ventilatory effort continues and increases in an attempt to overcome the obstruction, shown by an increase in esophageal pressure change. Rib cage and abdominal movements are in the opposite direction as a result of the diaphragm contracting against an occluded airway, forcing the abdominal wall to distend out and the chest wall to cave inward.

Increasing efforts to breathe lead to an arousal from sleep, visualisable on an EEG (FIG. 1), and result in opening of the airway and a resumption of normal breathing. The lack of airflow during the apnea also causes hypoxia, shown by a drop in oxyhemoglobin saturation (FIG. 1). Severity is generally measured using the apnea-hypopnea index (AHI), which is the combined average number of apneas (cessation of breathing for at least ten seconds) and hypopneas (reduced airflow and oxygen saturation) that occur per hour of sleep (Ruehland, W R. et al., The new AASM criteria for scoring hypopneas: Impact on the apnea hypopnea index. SLEEP 2009; 32 (2): 150-157).

FIG. 1 is a graphic illustration of an obstructive apnea. The top channel shows the electroencephalogram (EEG) pattern of sleep. The next channel represents airflow. The next three channels show ventilatory effort by movements of the rib cage and abdomen and changes in esophageal pressure, all of which reflect a respiratory effort against an occluded upper airway. The last channel indicates oxyhemoglobin saturation.

When a stringent definition of OSA is used (an AHI of >15 events per hour or AHI>5 events per hour with daytime sleepiness), the estimated prevalence is approximately 15 percent in males and 5 percent in females. An estimated 30 million individuals in the United States have OSA, of which approximately 6 million have been diagnosed. The prevalence of OSA in the United States appears to be increasing due to aging and increasing rates of obesity. OSA is associated with major comorbidities and economic costs, including: hypertension, diabetes, cardiovascular disease, motor vehicle accidents, workplace accidents, and fatigue/lost productivity. (Young, T. et al., WMJ 2009; 108:246; Peppard, P E. et al., Am J Epidemiol 2013; 177:1006.)

The present leading treatment is continuous positive airway pressure (CPAP). CPAP is effective in virtually all patients, and approximately 85% of diagnosed patients are prescribed CPAP, but compliance is low. Patients find CPAP uncomfortable and often intolerable; at least 30% of patients (up to 80%) are regularly non-adherent and thus untreated (Weaver, T E. Proc Am Thorac Soc. 2008 Feb. 15; 5 (2): 173-178). Other treatment modalities with variable rates of success include oral appliances (10%) and surgery (5%), but neither is likely to be effective across the general population.

The search for medicines to activate pharyngeal muscles in sleeping humans has been discouraging; agents such as serotonin reuptake inhibitors, tricyclic antidepressants, and sedatives have all been tested in humans and shown to be ineffective at reducing OSA severity. See, e.g., Hudgel, D A. et al., Chest. 1991 August; 100 (2): 416-21; Brownell L G. et al., N Engl J Med 1982, 307:1037-1042; Sangal R B. et al., Sleep Med. 2008 July; 9 (5): 506-10. Epub 2007 Sep. 27; Marshall, N S. et al. Sleep 2008 June; 31 (6): 824-31; Eckert, D J. et al., Clin Sci (Lond). 2011 June; 120 (12); 505-14; Taranto-Montemurro, L. et al., Sleep 2017 Feb. 1; 40 (2): ZSW047.

In a recent study, a combination of atomoxetine and oxybutynin, referred to as “ato-oxy,” administered before bedtime has been shown to reduce OSA in patients with a wide range of severity. The ato-oxy combination, which was administered for one night, reduced the number of obstructive events, improved the overnight oxygen desaturation, and enhanced the genioglossus muscle activity in a group of unselected patients with OSA. The data collected in the proof-of-concept trial showed that it was possible to improve or abolish OSA using drugs with specific neurotransmitter profiles administered systemically. See Taranto-Montemurro, L. et al., The Combination of Atomoxetine and Oxybutynin Greatly Reduces Obstructive Sleep Apnea Severity. A Randomized, Placebo-controlled, Double-Blind Crossover Trial. Am J Respir Crit Care Med 2019 May 15; 199 (10): 1267-1276.

There remains a need for further therapies for treating conditions associated with pharyngeal airway collapse such as sleep apnea.

### Methods of Treatment

The methods described herein include the treatment of disorders associated with pharyngeal airway muscle collapse during sleep. In some embodiments, the disorder is sleep apnea (e.g., obstructive sleep apnea (OSA)) or snoring (e.g., simple snoring). Generally, the methods include administering a therapeutically effective amount of a norepinephrine reuptake inhibitor (NRI) in combination with mandibular advancement device (MAD) therapy, to a subject who is in need of, or who has been determined to be in need of, such treatment. In some embodiments, the treatment further comprises administering a muscarinic receptor antagonist (MRA) and/or a hypnotic. In certain embodiments, the methods include administering a therapeutically effective amount of (i) atomoxetine or a pharmaceutically acceptable salt thereof and (ii) oxybutynin (e.g., (R)-oxybutynin) or a pharmaceutically acceptable salt thereof, in combination with mandibular advancement device (MAD) therapy, to a subject who is in need of, or who has been determined to be in need of, such treatment. In certain embodiments, the methods include administering a therapeutically effective amount of (i) atomoxetine or a pharmaceutically acceptable salt thereof and (ii) a hypnotic (e.g., trazodone or zolpidem or a pharmaceutically acceptable salt thereof), in combination with mandibular advancement device (MAD) therapy, to a subject who is in need of, or who has been determined to be in need of, such treatment.

As used in this context, to “treat” means to ameliorate at least one symptom of the disorder associated with pharyngeal airway collapse. Often, pharyngeal airway collapse during sleep results in snoring and/or an interruption in breathing (apnea or hypopnea), arousal from sleep, and reduced oxygenation (hypoxemia); thus, a treatment can result in a reduction in snoring, apneas/hypopneas, sleep fragmentation, and hypoxemia. Administration of a therapeutically effective amount of a compound described herein for the treatment of a subject with OSA may result in decreased AHI. Measurement of OSA disease and symptoms may be, for example, by polysomnography (PSG).

In general, an “effective amount” of a compound refers to an amount sufficient to elicit the desired biological response, e.g., to treat a condition associated with pharyngeal airway collapse, e.g., to treat sleep apnea or snoring. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

An effective amount can be administered in one or more administrations, applications or dosages. The NRI, MRA and/or hypnotic can be administered from one or more times per day to one or more times per week; including once every other day. In some embodiments, the the NRI, MRA and/or hypnotic are administered daily. In some embodiments, the NRI, MRA and/or hypnotic are administered daily before sleep time, e.g., immediately before sleep time or 15-60 minutes before sleep time. In some embodiments, the NRI, MRA and/or hypnotic are administered daily before placing the MAD in the subject, e.g., immediately before placing the MAD or 15-60 minutes before placing the MAD. In some embodiments, the NRI, MRA and/or hypnotic are administered daily concurrently with the MAD already placed in the subject. In some embodiments, the NRI, MRA and/or hypnotic are administered as a single composition. In some embodiments, the compositions are administered orally. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, “pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salts” includes “pharmaceutically acceptable acid addition salts” and “pharmaceutically acceptable base addition salts.” “Pharmaceutically acceptable acid addition salts” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salts” include those derived from inorganic bases such as sodium, potassium, lithium, ammonium, calcium, magnesium, iron, zinc, copper, manganese, aluminum salts, and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines, and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, Berge, S M. et al., “Pharmaceutical Salts,” J. Pharm. Sci., 1977; 66:1-19 which is incorporated herein by reference.)

As used herein, the term “unit dosage form” is defined to refer to the form in which the compound is administered to a subject. Specifically, the unit dosage form can be, for example, a pill, capsule, or tablet. In some embodiments, the unit dosage form is a capsule. In some embodiments, the unit dosage form is a tablet.

As used herein, “solid dosage form” means a pharmaceutical dose(s) in solid form, e.g. tablets, capsules, granules, powders, sachets, reconstitutable powders, dry powder inhalers and chewables.

For the compounds disclosed herein, single stereochemical isomers, as well as enantiomers, diastereomers, cis/trans conformation isomers, and rotational isomers, and racemic and non-racemic mixtures thereof, are within the scope of the invention. Unless otherwise indicated, all tautomeric forms of the compounds disclosed herein are within the scope of the invention.

### Norepinephrine Reuptake Inhibitors (NRIs), Muscarinic Receptor Antagonists (MRAs), and Hypnotics

Exemplary norepinephrine reuptake inhibitors (NRIs) include the selective NRIs, e.g., amedalin (UK-3540-1), atomoxetine (Strattera), CP-39,332, daledalin (UK-3557-15), edivoxetine (LY-2216684), esreboxetine, lortalamine (LM-1404), nisoxetine (LY-94,939), reboxetine (Edronax, Vestra), talopram (Lu 3-010), talsupram (Lu 5-005), tandamine (AY-23,946), viloxazine (Vivalan); and the non-selective NRIs, e.g., amitriptiline, amoxapine, bupropion, ciclazindol, desipramine, desvenlafaxine, dexmethilphenidate, diethylpropion, doxepin, duloxetine, imipramine, levomilnacipran, manifaxine (GW-320,659), maprotiline, methylphenidate, milnacipran, nefazodone, nortriptyline, phendimetrazine, phenmetrazine, protryptyline, radafaxine (GW-353,162), tapentadol (Nucynta), teniloxazine (Lucelan, Metatone) and venlafaxine; and pharmaceutically acceptable salts thereof.

In some embodiments, the NRI is atomoxetine or a pharmaceutically acceptable salt thereof. In some embodiments, the NRI is reboxetine or a pharmaceutically acceptable salt thereof.

Atomoxetine is the generic name of the pharmaceutical substance with the chemical name (−)-N-Methyl-3-phenyl-3-(o-tolyloxy)-propylamine, and its pharmaceutical salts. Atomoxetine is the R (−)-isomer as determined by x-ray diffraction. In some embodiments, atomoxetine may be atomoxetine hydrochloride.

In some embodiments, the methods include administering a dose of from about 20 mg to about 150 mg of atomoxetine or a pharmaceutically acceptable salt thereof (or a dose equivalent of another NRI). In some embodiments, the dose of atomoxetine or a pharmaceutically acceptable salt thereof is from about 25 mg to about 100 mg. In some embodiments, the dose of atomoxetine or pharmaceutically acceptable salt thereof is from about 40 mg to about 80 mg. In some embodiments, the dose of atomoxetine or pharmaceutically acceptable salt thereof is from about 20 mg to about 50 mg. In some embodiments, the dose of atomoxetine or a pharmaceutically acceptable salt thereof is from about 50 mg to about 100 mg. In some embodiments, the dose of atomoxetine or pharmaceutically acceptable salt thereof is about 40 mg. In some embodiments, the dose of atomoxetine or pharmaceutically acceptable salt thereof is about 80 mg.

Exemplary muscarinic receptor antagonists (MRAs) include atropine, propantheline, bethanechol, solifenacin, darifenacin, tolterodine, fesoterodine, trospium, and oxybutynin, and pharmaceutically acceptable salts thereof, which have activity on the M2 receptor. Other exemplary antimuscarinics include anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, and pharmaceutically acceptable salts thereof.

In some embodiments, the muscarinic receptor antagonist is oxybutynin or (R)-oxybutynin, or a pharmaceutically acceptable salt thereof. As used herein, (R)-oxybutynin refers to the (R)-oxybutynin stereoisomer substantially free of other stereoisomers of oxybutynin. In some embodiments, the muscarinic receptor antagonist is fesoterodine.

Oxybutynin is the generic name for the pharmaceutical substance with the chemical name 4-diethylamino-2-butynylphenylcyclohexylglycolate or 4-(diethylamino) but-2-ynyl 2-cyclohexyl-2-hydroxy-2-phenylacetate, and its pharmaceutically acceptable salts. In various embodiments, oxybutynin may be a racemic mixture of R- and S-enantiomers, or an isolated enantiomer, e.g., the R-enantiomer. In various embodiments, oxybutynin may be oxybutynin chloride or (R)-oxybutynin chloride.

In methods comprising administration of oxybutynin or (R)-oxybutynin or a pharmaceutically acceptable salt thereof (or another MRA), the dose of oxybutynin or (R)-oxybutynin or pharmaceutically acceptable salt thereof may be from about 0.5 mg to about 25 mg (or a dose equivalent thereof of another MRA), or in some embodiments, from about 2 mg to about 15 mg. In some embodiments, the dose of oxybutynin or pharmaceutically acceptable salt thereof is from about 2.5 mg to about 10 mg, e.g., 5 mg. In some embodiments, the dose of (R)-oxybutynin or pharmaceutically acceptable salt thereof is from about 0.5 mg to about 5 mg, e.g., 2.5 mg. In some embodiments, the dose of oxybutynin or (R)-oxybutynin or pharmaceutically acceptable salt thereof is from about 1 mg to about 5 mg.

Exemplary hypnotics include benzodiazepines, e.g., temazepam, brotizolam, flurazepam, nitrazepam, and triazolam; cyclopyrrolone hypnotics, e.g., zolpidem, zopiclone, and eszopiclone; gabapentin; trazodone; diphenhydramine; suvorexant; tasimelteon; ramelteon; agomelatine; doxepin; zaleplon; doxylamine; sodium oxybate; and tiagabin and pharmaceutically acceptable salts thereof.

In some embodiments, the hypnotic is trazodone or a pharmaceutically acceptable salt thereof. In some embodiments, the hypnotic is zolpidem or a pharmaceutically acceptable salt thereof.

### Mandibular Advancement Devices (MADs)

Mandibular Advancement Devices (MADs), including mandibular advancement splints (MAS) or mandibular repositioning appliances (MRA), prevent upper airway collapse by protruding the mandible forward, thus altering the jaw and tongue position. Both video endoscopy and magnetic resonance imaging (MRI)-guided studies have determined that these devices predominantly increase the volume of the airway at the level of the velopharynx. The airway space is mostly enlarged laterally, thought to be due to traction on soft tissue connections between the pharynx and the mandibular ramus. MADs also improve the strength and rigidity of the airway by increasing the muscle activity of the tongue and other muscles of the airway.

The design and sophistication of MADs vary greatly. Variables include adjustability, nature or extent of customization and materials used; and they are not mutually exclusive.

In some embodiments, the MAD is a boil and bite MAD, a one-piece custom MAD, or a two-piece custom MAD.

In some embodiments, the MAD is a boil and bite MAD. Non-adjustable, “boil and bite” MADs can be obtained from pharmacies and on various websites. They are constructed of a thermoplastic material that becomes moldable when warmed by immersion in hot water. The user takes a mold of their teeth by biting into the softened material that then sets on cooling.

In some embodiments, the MAD is a custom made MAD. Custom made MADs are constructed in a lab using dental impressions. Custom made MADs can be a one-piece or an adjustable two-piece device.

In some embodiments, the MAD is a one-piece custom MAD. Upper and lower dental splints are fused in the one-piece device (monobloc). Although most of these appliances are a bespoke dentally produced device, “semi-bespoke” MAD, which require no specialist dental input, exist.

In some embodiments, the MAD is a two-piece custom MAD. Adjustable two-piece devices come in separate upper and lower plates. Construction requires additional specialist jaw articulation and is more expensive. Serially titrated mandibular protrusion is thought to increase treatment success by allowing gradual adaptation to optimal protrusion. The ability to titrate protrusion according to efficacy and tolerance is an advantage of adjustable MAD (aMAD). Existing studies that have compared so-called fixed MAD (fMAD) to aMAD have had methodological limitations and inconsistent findings. For example, one study comparing two devices set different protrusions for fMADs and aMADs, thus essentially comparing protrusions rather than devices. Accordingly, the MAD to be used in the methods of the present invention can vary depending on the subject's needs and body/mouth/teeth structure.

### Pharmaceutical Compositions

Also provided herein are pharmaceutical compositions comprising a norepinephrine reuptake inhibitor (NRI) and a muscarinic receptor antagonist (MRA) and/or a hypnotic, as active ingredients. The active ingredients can be in a single composition or in separate compositions. In certain embodiments, the pharmaceutical compositions include (i) atomoxetine or a pharmaceutically acceptable salt thereof and (ii) oxybutynin (e.g., (R)-oxybutynin) or a pharmaceutically acceptable salt thereof, as active ingredients.

Pharmaceutical compositions typically include a pharmaceutically acceptable carrier. As used herein the language “pharmaceutically acceptable carrier” includes saline, solvents, dispersion media, diluents, fillers, coatings, antibacterial and antifungal agents, isotonic and absorption delaying agents, and the like, compatible with pharmaceutical administration.

The active ingredients for use in the present invention may be provided as pharmaceutically acceptable salts. For example, in some embodiments, oxybutynin is oxybutynin chloride. In some embodiments, (R)-oxybutynin is (R)-oxybutynin chloride. In some embodiments, atomoxetine is atomoxetine hydrochloride.

Pharmaceutical compositions are typically formulated to be compatible with its intended route of administration. Examples of routes of administration include systemic oral or transdermal administration, as well as sublingual administration, e.g., via tablet or spray.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, oral compositions generally include an inert diluent or an edible carrier. For the purpose of oral therapeutic administration, the active compound(s) can be incorporated with excipients and used in the form of pills, tablets, troches, or capsules, e.g., gelatin capsules. Oral compositions can also be prepared using a fluid carrier. In some embodiments, a composition according to the present invention may be a unit dosage form. In some embodiments, a composition according to the present invention may be a solid dosage form, e.g., a tablet or capsule.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration of the compounds as described herein can also be by transdermal means, e.g., using a patch, gel, or lotion, to be applied to the skin. For transdermal administration, penetrants appropriate to the permeation of the epidermal barrier can be used in the formulation. Such penetrants are generally known in the art. For example, for transdermal administration, the active compounds can formulated into ointments, salves, gels, or creams as generally known in the art. The gel and/or lotion can be provided in individual sachets, or via a metered-dose pump that is applied daily; see, e.g., Cohn et al., Ther Adv Urol. 2016 April; 8 (2): 83-90.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration or use in a method described herein.

In some embodiments, the pharmaceutical composition is for use in treating a condition associated with pharyngeal airway collapse. In some embodiments, the condition is sleep apnea (e.g., OSA) or snoring (e.g., simple snoring). In certain embodiments, provided herein is a pharmaceutical composition comprising atomoxetine or a pharmaceutically acceptable salt thereof and cannabidiol or a pharmaceutically acceptable salt thereof, and optionally oxybutynin (e.g., (R)-oxybutynin) or a pharmaceutically acceptable salt thereof for use in treating sleep apnea (e.g., OSA) or snoring (e.g., simple snoring).

### Combinations

Also provided herein is a norepinephrine reuptake inhibitor (NRI) and a mandibular advancement device (MAD), for use in treating a subject having a condition associated with pharyngeal airway collapse. In some embodiments, the combination for use further comprises a muscarinic receptor antagonist (MRA). In some embodiments, the combination for use further comprises a hypnotic. Further provided herein is a therapeutic combination comprising (a) a pharmaceutical composition comprising a norepinephrine reuptake inhibitor (NRI) and a pharmaceutically acceptable carrier, and (b) a mandibular advancement device (MAD) for use in treating a subject having a condition associated with pharyngeal airway collapse. In some embodiments, the combination for use further comprises a muscarinic receptor antagonist (MRA). In some embodiments, the combination for use further comprises a hypnotic. Various embodiments of combinations and therapeutic combinations will be apparent from the detailed description provided herein, including from the compositions and methods described herein. In certain embodiments of the combinations of the present invention, the NRI is atomoxetine or a pharmaceutically acceptable salt thereof and the MRA is oxybutynin (e.g., (R)-oxybutynin) or a pharmaceutically acceptable salt thereof.

### EXAMPLES

The invention is further described in the following examples, which do not limit the scope of the invention described in the claims.

### Example 1. Randomized, Double-Blind, Multiple-Dose 2-Period Crossover Study to Evaluate the Efficacy and Safety of AD036 (Atomoxetine+Oxybutynin) Plus Mandibular Advancement Device Vs. AD036 Alone in OSA Patients with Suboptimal Response to Mandibular Advancement Device. (MandADO)

### Rationale

AD036, a combination of atomoxetine and oxybutynin, is a drug combination under development for the treatment of obstructive sleep apnea (OSA). The primary mechanism of action of AD036 is thought to be increased pharyngeal muscle stiffness and responsiveness.

Mandibular advancement device (MAD) therapy improves OSA by mechanically increasing the retropalatal and retrolingual spaces. Improvement of OSA severity from MAD therapy, as measured by apnea-hypopnea index (AHI), is typically about 50%, but in some patients improvement may be less, or may be considered inadequate, e.g. because of elevated residual AHI or subjective reports of continued excessive daytime sleepiness (EDS) or snoring.

The MandADO study is designed to assess the safety and efficacy for OSA of combination treatment with AD036 and MAD in patients with inadequate response to MAD alone.

### Obstructive Sleep Apnea

The National Commission on Sleep Disorders Research identified sleep disorders as a major public health burden. OSA is the most common and serious of these sleep disorders and affects approximately 20 million people in the United States (US), with approximately 13% of men and 6% of women affected (1). OSA is characterized by repetitive collapse or ‘obstruction’ of the pharyngeal airway during sleep, manifestingas repetitive episodes of hypopnea (i.e., shallow breathing) or apnea (i.e., paused breathing). These episodes of hypopnea or apnea may lead to arousal from sleep, sleep fragmentation, excessive daytime sleepiness, and/or neuropsychological impairment.

Research has shown that a number of pathogenic factors, or traits, contribute to the development of OSA (2-5). The most important factors are the presence of an anatomically small, collapsible upper airway and a loss of pharyngeal muscle tone or responsiveness during sleep.

Long-term, OSA is associated with increased mortality and a number of adverse cardiovascular, neurocognitive, metabolic, and daytime functioning consequences (6-15).

### Unmet Medical Need

The most common treatment for OSA is currently positive airway pressure, typically continuous positive airway pressure (CPAP) provided by a device that mechanically maintains an open airway. While efficacy of CPAP is often satisfactory when the device is used, many, perhaps most, patients find these devices uncomfortable or intolerable, and most estimates indicate that fewer than 50% of patients prescribed CPAP use it more than 4 hours per night, if at all (16). Mandibular advancement devices are an alternative to CPAP, but patients may have a suboptimal treatment response. Current pharmacologic therapies are limited to treatment of excessive daytime sleepiness from OSA.

Study Endpoints are shown in the table 1 below.

TABLE 1
Study Endpoints.

| | Endpoints |
|---|---|
| Primary | AHI4%, MAD + AD036 vs MAD alone |
| Secondary | HB4%, ODI4%, Total time with SaO₂ <90%, Proportion of |
| | participants with ≥50% reduction |
| | in AHI4%, HB4%, ODI4% |
| Exploratory | PGI-S |
| | PROMIS sleep impairment |
| | PROMIS sleep disturbance |
| | PROMIS fatigue |
| | AHI3a (hypopnea scored when associated |
| | with 3% O₂ desaturation or arousal) |
| | OSA endotype endpoints (Vpassive, Vactive, |
| | Muscle Compensation, Loop Gain) |
| | PSG sleep and arousal parameters |
| Safety | Physical exam, vital signs, clinical |
| Endpoints | laboratory assessment |
| | Spontaneous adverse events |

Abbreviations:
AHI = apnea-hypopnea index;
HB = hypoxic burden;
MAD = mandibular advancement device;
ODI = Oxygen Desaturation Index;
OSA = obstructive sleep apnea;
PROMIS = Patient Reported Outcome Measurement Information System;
PGI-S = Patient Global Impression of Severity;
PROMIS = Patient Reported Outcome Measurement Information System;
SaO₂ = oxygen saturation;

### Overall Study Design

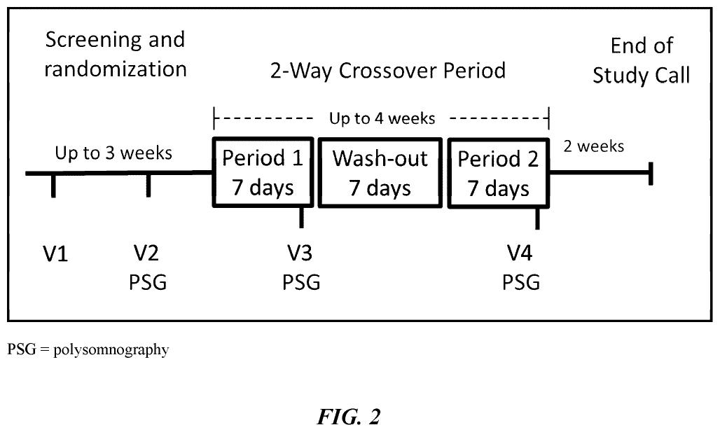

FIG. 2 provides an overview of the study design.

The MandADO study is a randomized, double-blind, placebo-controlled, 2-period crossover study in patients with inadequate response to MAD alone. Patients with elevated residual AHI or subjective reports of EDS or snoring on current custom-made MAD therapy provided by a dental or maxillofacial specialist are eligible for screening if there is clinical suspicion or evidence of elevated residual AHI. Participants will undergo initial pre-screening to determine potential study eligibility or exclusionary factors, followed by screening Visit 1 for patients who remain eligible. Only participants who subsequently meet all non-PSG enrollment criteria at Visit 1 are eligible for a screening PSG at Visit 2. The screening PSG is conducted with the MAD in place. Patients are eligible for enrollment in the study if the residual AHI (4%) with the MAD is >10 and all other enrollment criteria are met.

Enrolled patients will be randomized for 1-week periods each the following two study treatments:
Period 1: MAD use nightly on all nights, combined with a low-dose run-in period of AD036 on Days 1-3 consisting of 40 mg atomoxetine and 5 mg oxybutynin, followed by a full dose period of AD036 on Days 4-7 consisting of 80 mg atomoxetine and 5 mg oxybutynin (all doses over-encapsulated).
Period 2: MAD used nightly on all nights, combined with 2 matching placebo capsules.

Study drug for Period 1 is dispensed at Visit 2 prior to patient discharge. The study drug consists of two different tablets, one of each of which is taken each night at the patient's usual bedtime. Following 6 (up to 8) days of at-home dosing, patients return with the remaining dispensed study drug for PSG at Visit 3, with dosing at lights out from that drug supply. The morning after each PSG the symptom questionnaires are administered, and study drug for the second crossover period is dispensed. Patients are instructed not to begin taking the study drug for the second period until after the 1-week washout period. At the conclusion of the 1-week washout period, the site contacts that patient by phone to initiate dosing of the second crossover period. Similar to Period 1, following 6 (up to 8) days of at-home dosing, patients return with the remaining dispensed study drug for PSG at Visit 4, with dosing of study drug at Visit 4 from the patient's Period 2 supply.

Adverse event and concomitant medication information is collected at each study site visit and two weeks after Visit 4 during the End-of-Study call. The End-of-Study call with the patient will take place 2 weeks following the end of study drug dosing.

Participants who discontinue from the study will not be replaced. No subsequent open-label extension is planned following the study.

### Inclusion Criteria

1. Current use of MAD for OSA; patients who discontinued MAD within 6 months are eligible if MAD use is restarted at least 2 weeks prior to V2
2. 25 to 65 years of age, inclusive, at the Screening Visit.
3. AHI (4%)>10 at V2 baseline with use of MAD.
   If AHI (4%) is 8-9 on initial PSG, can be repeated and average AHI (4%) used
4. ≤25% of apneas are central or mixed apneas at V2 baseline PSG
5. BMI between 18.5 and 40.0 kg/m², inclusive, at the pre-PSG screening visit
6. If male and sexually active with female partner(s) of childbearing potential, participant must agree, from Study Day 1 through 1 week after the last dose of study drug, to practice the protocol specified contraception.
7. If a woman of childbearing potential (WOCBP), the participant must agree, from Study Day 1 through 1 week after the last dose of study drug, to practice the protocol specified contraception (See Appendix 4: Contraceptive Guidance and Collection of Pregnancy Information). All WOCBP must have negative result of a serum pregnancy test performed at screening.
8. If female and of non-childbearing potential, the participant must be either postmenopausal (defined as age≥55 years with no menses for 12 or more months without an alternative medical cause) or permanently surgically sterile (bilateral oophorectomy, bilateral salpingectomy or hysterectomy).
9. Participant voluntarily agrees to participate in this study and signs an Institutional Review Board (IRB)-approved informed consent prior to performing any of the Screening Visit procedures.
10. Participant must be able to understand the nature of the study and must have the opportunity to have any questions answered.

### Exclusion Criteria

Participants are excluded from the study if any of the following criteria apply:
1. History of clinically significant sleep disorder other than OSA.
2. Clinically significant craniofacial malformation.
3. Clinically significant cardiac disease (e.g., rhythm disturbances, coronary artery disease or cardiac failure) or hypertension requiring more than 2 medications for control (combination medications are considered as 1 medication for this purpose).
4. Clinically significant neurological disorder, including epilepsy/convulsions.
5. History of schizophrenia, schizoaffective disorder or bipolar disorder according to Diagnostic and Statistical Manual of Mental Disorders-5 (DSM-5) or International Classification of Disease tenth edition criteria.
6. History of attempted suicide within 1 year prior to screening, or current suicidal ideation.
7. Medically unexplained positive screen for drugs of abuse or history of substance use disorder as defined in DSM-V within 24 months prior to Screening Visit.
8. A significant illness or infection requiring medical treatment in the past 30 days.
9. Clinically significant cognitive dysfunction as determined by investigator.
10. Women who are pregnant or nursing.
11. Use of positional devices is allowed but should be kept constant in the 2 weeks prior to V2 and during the course of the trial.
12. CPAP use must be discontinued at least 2 weeks prior to V2 and during the course of the trial.
13. History of chronic oxygen therapy.
14. Use of medications from the list of disallowed concomitant medications.
15. Treatment with strong cytochrome P450 3A4 (CYP3A4) inhibitors, strong cytochrome P450 2D6 (CYP2D6) inhibitors, or monoamine oxidase inhibitors (MAOI) within 14 days of the start of treatment, or concomitant with treatment.
16. Use of another investigational agent within 30 days or 5 half-lives, whichever is longer, prior to dosing.
17. Hepatic transaminases>2× the upper limit of normal (ULN), total bilirubin>1.5×ULN (unless confirmed Gilbert syndrome), estimated glomerular filtration rate<60 ml/min.
18. PLM arousal index>15
19. <5 hours typical sleep duration.
20 ESS>18
21. Night- or shift-work sleep schedule which causes the major sleep period to be during the day.
22. Employment as a commercial driver or operator of heavy or hazardous equipment.
23. Typically smoking more than 10 cigarettes or 2 cigars per day, or inability to abstain from smoking during overnight PSG visits.
24. Unwilling to use specified contraception.
25. History of regular alcohol consumption of more than 14 standard units per week (males) or more than 7 standard units per week (females), or unwillingness to limit alcohol consumption to no greater than 2 units/day (males), 1 unit per day (females), not to be consumed within 3 hours of bedtime or on PSG nights.
26. Unwilling to limit during the study period caffeinated beverage intake (e.g., coffee, cola, tea) to 400 mg/day or less of caffeine, not to be used within 3 hours of bedtime.
27. Any condition that in the investigator's opinion would present an unreasonable risk to the participant, or which would interfere with their participation in the study or confound study interpretation.
28. Participant considered by the investigator, for any reason, an unsuitable candidate to receive atomoxetine and/or dronabinol or unable or unlikely to understand or comply with the dosing schedule or study evaluations.

### Meals and Dietary Restrictions

1. Participants should refrain from consumption of any nutrients known to modulate CYP enzyme activity (e.g., grapefruit or grapefruit juice, pomelo juice, star fruit, pomegranate, and Seville or Moro [blood] orange products) within 72 hours before the first dose of study drug and during the study.
2. Diet should be generally stable during the study, e.g., new diet programs should not be initiated.

### Caffeine, Alcohol, and Tobacco

1. During the outpatient portions of the study, participants should refrain from more than 2 standard units per day of alcohol for men or 1 unit/day for women, consumed no less than 3 hours prior to bedtime. Alcohol should not be consumed on PSG nights.
2. Moderate consumption of caffeinated beverages, containing up to a total of 400 mg caffeine per day, is permitted during the study period, consumed no less than 3 hours prior to bedtime.

### Study Drug

AD036 or placebo is taken in each crossover period, in combination with MAD. AD036 consists of one over-encapsulated atomoxetine (40 mg days 1-3, 80 mg days 4-7) and one over-encapsulated oxybutynin 5 mg. Table 2 shows the dosage formulation and routes of administration.

TABLE 2
Dosage Formulation

| | Study Treatment Name: | | |
|---|---|---|---|
| | Atomoxetine | | |
| | hydrochloride | Oxybutynin | Placebo |
| Dosage Formulation: | Capsule | Tablet | Capsule |
| Dosage Levels: | 40 mg, 80 mg | 5 mg | n/a |
| Route of | Oral | Oral | Oral |
| Administration: | | | |
| Dosing | 1 capsule | 1 capsule | 1 capsule |
| Instructions: | administered | administered | administered |
| | with up to | with up to | with up to |
| | 240 mL water | 240 mL water | 240 mL water |
| Storage/Packaging/ | Store at room | Store at room | Store at room |
| Labeling: | temperature, in | temperature in | temperature in |
| | HDPE bottles | HDPE bottles | HDPE bottles |

### Concomitant Therapy

Concomitant therapy with the following medications listed below is disallowed. For medication that is typically used as-needed for symptomatic conditions (e.g., occasional use of a sleep aid), the medication should not be used for at least one week prior to the first study PSG and for the duration of the study.

Medications not allowed include, MAOIs or other drugs that affect monoamine concentrations (e.g., rasagiline) [MAOIs are contraindicated for use with Atomoxetine; Lithium; Cannabinoids; Selective Serotonin Reuptake Inhibitors (e.g., paroxetine); Selective Norepinephrine Reuptake Inhibitors (e.g., duloxetine); Norepinephrine Reuptake Inhibitors (e.g., reboxetine); Alpha-1 antagonists (e.g., tamsulosin); Tricyclic antidepressants (e.g., desipramine); CYP2D6 inhibitors; Strong CYP3A4 inhibitors (e.g., ketoconazole); Benzodiazepines and other anxiolytics; Opioids; Sedatives and sedative-hypnotics, including nonbenzodiazepine “Z-drugs” (zolpidem, zaleplon, eszopiclone); Muscle relaxant; Pressor agents; Drugs with clinically significant cardiac QT-interval prolonging effects; Drugs known to lower seizure threshold (e.g., chloroquine); Amphetamines; Antiepileptics; Antiemetics; Modafinil or armodafinil; Beta2 agonists, (e.g., albuterol); Antipsychotics; Sedating antihistamines; Pseudoephedrine, phenylephrine, oxymetazoline; Nicotine replacement products; Most drugs for Parkinson's, Alzheimer's, Huntington's, Amyotrophic Lateral Sclerosis, or drugs for other neurodegenerative diseases.

Medications that do not have substantial effects on the central nervous system (CNS), respiration, or muscle activity are generally allowed including, but not necessarily limited to, the following drugs and drug classes: Antihypertensives (angiotensin-converting-enzyme [ACE]/angiotensin II receptor blocker [ARB] inhibitors, calcium channel blockers, hydrochlorothiazide, etc.); Statins; Proton pump inhibitors and histamine h2 receptor blockers; Over-the-counter (OTC) antacids; Non-sedating antihistamines (e.g., cetirizine, loratadine); Acetaminophen; Laxatives; Erectile dysfunction drugs; Inhaled corticosteroids (e.g., fluticasone); Anti-diabetics; Ocular hypotensives and other ophthalmics (e.g., timolol); Hormonal therapy (e.g., estrogen replacement or anti-estrogens) and hormonal contraceptives; Thyroid medications; Anticoagulants; Osteoporosis drugs.

### Study Assessments and Procedures

Study procedures and their timing are summarized in the SoA table shown in Table 3 below.

### Polysomnography

Methods: Standard overnight PSG recording and data interpretation will be performed in accordance with the American Academy of Sleep Medicine (AASM) scoring manual.

Participants will be instrumented with standard PSG electrodes. Time of lights out will be established according to the participants' habitual schedule and kept constant across the PSG study nights. The participants will be given 8 hours of time-in bed.

Participants should be actively encouraged to spend at least ⅓ of the night in the supine position and at least ⅓ of the night in the lateral position on each night of study.

### Safety Assessments

Planned time points for all safety assessments are provided in the SoA Table 3.

Safety monitoring will be guided by the established safety profiles of atomoxetine and oxybutynin, and MADs. Safety assessments will include physical examinations, measurement of vital signs, monitoring and recording of AEs, SAEs, and pregnancies, recording of study or treatment discontinuations. Effects on OSA and sleep parameters (e.g., sleep time and sleep stages) will also be monitored by PSG.

TABLE 3
Schedule of Activities.

| | Procedures | | | | | | | |
|---|---|---|---|---|---|---|---|---|
| | | | 2-Way Crossover | | | | | |
| | | | | 4 day¹ full dose or | | | 4 day¹ full dose or | |
| | | | 3 day run-in | placebo, | | 3 day run-in | placebo, | |
| | Screening | | dose or | followed by | Wash- | dose or | followed by | End of Study Call |
| Trial Day | V1 | V2 | placebo | in-lab PSG, V3 | out² | placebo | in-lab PSG, V4 | 2 weeks after V4 ± |
| (Visit Window) | Up to 3 weeks | | Up to 4 weeks | | | | | 3 d |
| Informed consent | X | | | | | | | |
| Demography | X | | | | | | | |
| Physical exam | X | | | | | | | |
| Medical history | X | | | | | | | |
| Pregnancy test³ | X | | | | | | | |
| Clinical | X | | | | | | | |
| laboratory testing | | | | | | | | |
| 12 Lead ECG | X | | | | | | | |
| PSG | | X | | X | | | X | |
| Randomization | | X⁴ | | | | | | |
| Study drug and device | | X | | X | | | X | |
| dispense/return | | | | | | | | |
| HS study treatment⁵ | | | X | X | | X⁶ | X | |
| ESS, Short SAQLI, | X | | | X⁷ | | | X⁷ | |
| PGI-S, PROMIS | | | | | | | | |
| sleep impairment, | | | | | | | | |
| PROMIS sleep | | | | | | | | |
| disturbance, | | | | | | | | |
| PROMIS fatigue | | | | | | | | |
| Vital signs⁸ | X | X | | X | | | X | |
| and body weight | | | | | | | | |
| AE/SAE monitoring | | | ↔ | ↔ | ↔ | ↔ | ↔ | ↔ |
| Prior/concomitant | ↔ | ↔ | ↔ | ↔ | ↔ | ↔ | ↔ | ↔ |
| medication | | | | | | | | |

¹Up to 6 days if necessary for scheduling
²Each washout period is a minimum of 7 days
³WOCBP only
⁴Randomization the morning after V2 PSG after eligibility confirmed
⁵Study medication administered immediately before lights out
⁶Site contacts patient by phone to initiate dosing
⁷Administer at similar time on evening of each crossover PSG, approximately 1 hour after admission
⁸Vital signs include the following: seated blood pressure, pulse, respiratory rate; vital signs on PSG nights taken evening of admission to PSG lab

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject having a condition associated with pharyngeal airway collapse, the method comprising administering to a subject in need thereof an effective amount of a norepinephrine reuptake inhibitor (NRI) in combination with mandibular advancement device (MAD) therapy.

2. The method of claim 1, wherein the NRI is a norepinephrine selective reuptake inhibitor (NSRI).

3. The method of claim 2, wherein the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof.

4. The method of claim 1, wherein the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof.

5. The method of claim 1, wherein the NRI is reboxetine or a pharmaceutically acceptable salt thereof.

6. The method of claim 1, wherein the NRI is atomoxetine or a pharmaceutically acceptable salt thereof.

7. The method of any one of claims 1-6, further comprising administering an effective amount of a muscarinic receptor antagonist (MRA).

8. The method of claim 7, wherein the MRA is selected from the group consisting of atropine, propantheline, bethanechol, solifenacin, darifenacin, tolterodine, fesoterodine, trospium, and oxybutynin, or a pharmaceutically acceptable salt thereof.

9. The method of claim 7, wherein the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof.

10. The method of claim 8, wherein the MRA is oxybutynin or a pharmaceutically acceptable salt thereof.

11. The method of claim 10, wherein the MRA is (R)-oxybutynin or a pharmaceutically acceptable salt thereof.

12. The method of any one of claims 1-11, wherein the atomoxetine or pharmaceutically acceptable salt thereof is administered at a dose of from about 20 to about 150 mg.

13. The method of claim 12, wherein the atomoxetine or pharmaceutically acceptable salt thereof is administered at a dose of from about 25 to about 100 mg.

14. The method of any one of claims 12-13, wherein the oxybutynin or pharmaceutically acceptable salt thereof is administered at a dose of from about 1 to about 15 mg.

15. The method of claim 14, wherein the oxybutynin or pharmaceutically acceptable salt thereof is administered at a dose of from about 2 mg to about 10 mg.

16. The method of any one of claims 12-13, wherein the (R)-oxybutynin or pharmaceutically acceptable salt thereof is administered at a dose of from about 0.5 to about 10 mg.

17. The method of claim 16, wherein the (R)-oxybutynin or pharmaceutically acceptable salt thereof is administered at a dose of from about 2 mg to about 6 mg.

18. The method of any one of claims 1-17, further comprising administering an effective amount of a hypnotic.

19. The method of claim 18, wherein the hypnotic is trazodone or a pharmaceutically acceptable salt thereof.

20. The method of claim 18, wherein the hypnotic is zolpidem or a pharmaceutically acceptable salt thereof.

21. The method of any one of claims 1-20, wherein the NRI, MRA, and/or hypnotic are administered in a single composition.

22. The method of claim 21, wherein the single composition is an oral administration form.

23. The method of claim 22, wherein the oral administration form is a syrup, pill, tablet, troche, capsule, or patch.

24. The method of claim 23, wherein the single composition is in an immediate release formulation.

25. The method of claim 24, wherein the NRI is atomoxetine or a pharmaceutically acceptable salt thereof and is administered at a dose of from about 20 to about 150 mg and the MRA is oxybutynin or a pharmaceutically acceptable salt thereof and is administered at a dose of from about 1 to about 15 mg.

26. The method of claim 25, wherein the NRI is atomoxetine or a pharmaceutically acceptable salt thereof and is administered at a dose of from about 25 to about 100 mg and the MRA is oxybutynin or a pharmaceutically acceptable salt thereof and is administered at a dose of from about 2 to about 10 mg.

27. The method of claim 25, wherein the NRI is atomoxetine or a pharmaceutically acceptable salt thereof and is administered at a dose of from about 20 to about 50 mg and the MRA is oxybutynin or a pharmaceutically acceptable salt thereof and is administered at a dose of from about 2 to about 10 mg.

28. The method of claim 25, wherein the NRI is atomoxetine or a pharmaceutically acceptable salt thereof and is administered at a dose of from about 50 to about 100 mg and the MRA is oxybutynin or a pharmaceutically acceptable salt thereof and is administered at a dose of from about 2 to about 10 mg.

29. The method of claim 23, wherein the single composition is in a controlled release formulation.

30. The method of claim 29, wherein the NRI is atomoxetine or a pharmaceutically acceptable salt thereof and is administered at a dose of from about 20 to about 150 mg and the MRA is oxybutynin or a pharmaceutically acceptable salt thereof and is administered at a dose of from about 0.5 to about 10 mg.

31. The method of claim 30, wherein the NRI is atomoxetine or a pharmaceutically acceptable salt thereof and is administered at a dose of from about 25 to about 100 mg and the MRA is oxybutynin or a pharmaceutically acceptable salt thereof and is administered at a dose of from about 1 to about 5 mg.

32. The method of claim 30, wherein the NRI is atomoxetine or a pharmaceutically acceptable salt thereof and is administered at a dose of from about 20 to about 50 mg and the MRA is oxybutynin or a pharmaceutically acceptable salt thereof and is administered at a dose of from about 2 to about 6 mg.

33. The method of claim 30, wherein the NRI is atomoxetine or a pharmaceutically acceptable salt thereof and is administered at a dose of from about 50 to about 100 mg and the MRA is oxybutynin or a pharmaceutically acceptable salt thereof and is administered at a dose of from about 2 to about 6 mg.

34. The method of any one of claims 21-33, wherein the single composition further comprises a pharmaceutically acceptable carrier.

35. The method of any one of claims 21-34, wherein the single composition is administered prior to the start of the mandibular advancement device (MAD) therapy.

36. The method of any one of claims 21-34, wherein the single composition is administered concurrently with the mandibular advancement device (MAD) therapy.

37. The method of any one of claims 1-36, wherein the condition associated with pharyngeal airway collapse is sleep apnea.

38. The method of claim 37, wherein the condition associated with pharyngeal airway collapse is obstructive sleep apnea (OSA).

39. The method of any one of claims 1-36, wherein the condition associated with pharyngeal airway collapse is snoring.

40. The method of claim 39, wherein the condition associated with pharyngeal airway collapse is simple snoring.

41. The method of any one of claims 1-40, wherein the subject is in a non-fully conscious state.

42. The method of claim 41, wherein the non-fully conscious state is sleep.

43. A norepinephrine reuptake inhibitor (NRI), a muscarinic receptor antagonist (MRA), and a mandibular advancement device (MAD), for use in treating a subject having a condition associated with pharyngeal airway collapse.

44. A norepinephrine reuptake inhibitor (NRI), a hypnotic, and a mandibular advancement device (MAD), for use in treating a subject having a condition associated with pharyngeal airway collapse.

45. A therapeutic combination comprising (a) a pharmaceutical composition comprising a norepinephrine reuptake inhibitor (NRI) and a pharmaceutically acceptable carrier, and (b) a mandibular advancement device (MAD) for use in treating a subject having a condition associated with pharyngeal airway collapse.

46. The therapeutic combination of claim 45, further comprising, in the pharmaceutical composition, a muscarinic receptor antagonist (MRA) and/or a hypnotic.

47. Atomoxetine or a pharmaceutically acceptable salt thereof, oxybutynin or a pharmaceutically acceptable salt thereof, and a mandibular advancement device (MAD), for use in treating a subject having a condition associated with pharyngeal airway collapse.

48. Atomoxetine or a pharmaceutically acceptable salt thereof, oxybutynin or a pharmaceutically acceptable salt thereof, and a mandibular advancement device (MAD), for use in treating sleep apnea.

49. Atomoxetine or a pharmaceutically acceptable salt thereof, oxybutynin or a pharmaceutically acceptable salt thereof, and a mandibular advancement device (MAD), for use in treating snoring.
